(19) United States
(12) Patent Application Publication
(10) Pub. No.: US 2016/0279674 A1
(43) Pub. Date: Sep. 29, 2016

(54) PROCESSING MINED MATERIAL

(71) Applicant: TECHNOLOGICAL RESOURCES PTY. LIMITED, Queensland (AU)

(72) Inventors: Samuel KINGMAN, Burton on Trent (GB); Christopher DODDS, Nottingham (GB); Paula PALADE, Beeston (GB); Georgios DIMITRAKIS, Nottingham (GB); Rebecca JOHN, Nottinghamshire (GB)

(73) Assignee: TECHNOLOGICAL RESOURCES PTY. LIMITED, Brisbane (AT)

(21) Appl. No.: 14/778,187

(22) PCT Filed: Mar. 19, 2014
(86) PCT No.: PCT/AU2014/000296

§ 371 Date: Sep. 18, 2015

(30) Foreign Application Priority Data
Mar. 20, 2013 (AU) 2013900972

(51) Int. Cl. B07C 5/344; G01N 27/02; G01N 25/72; B07C 5/36; G01N 33/24

(52) U.S. Cl. CPC B07C 5/344; B07C 5/363; G01N 33/24; G01N 25/72; G01N 27/025

(57) ABSTRACT

A method of processing mined material, such as mined ore, includes a step of exposing mined material to an alternating magnetic field and directly or indirectly assessing the electrical conductivity of mined material in the alternating magnetic field in order to determine whether there is valuable material in the mined material. An apparatus for processing mined material, such as mined ore, includes a magnetic field station that is adapted to expose mined material to an alternating magnetic field that induces currents in mined material that are related to the electrical conductivity of valuable material in the mined material.

### TECHNICAL FIELD

The present invention relates to recovering valuable material from mined material.

In particular, the present invention relates to a method of and an apparatus for processing mined material by direct or indirect assessment of the electrical conductivity of mined material.

In particular, the present invention relates to a method of and an apparatus for processing mined material for recovering valuable material from mined material by direct or indirect assessment of the electrical conductivity of mined material.

### BACKGROUND ART

The term “mined” material is understood herein to be any mined material that contains valuable material, such as valuable metals. Examples of valuable materials are valuable metals in minerals such as minerals that comprise metal oxides or metal sulphides. Specific examples of valuable materials that contain metal oxides are iron ores. Specific examples of valuable materials that contain metal sulphides are copper-containing ores. The term “mined” material is understood herein to include metalliferous material and non-metalliferous material. Iron-containing and copper-containing ores are examples of metalliferous material. Coal is an example of a non-metalliferous material. The term “mined” material is understood herein to include, but is not limited to, (a) run-of-mine material and (b) run-of-mine material that has been subjected to at least primary crushing or similar size reduction after the material has been mined and prior to being sorted. The term “mined” material includes mined material that is in stockpiles. The term “mined” material includes geological core samples.

A particular, although by no means exclusive, area of interest to the applicant is mined material in the form of mined ores that include valuable copper-containing sulphide minerals and non-valuable sulphide minerals that are often found in nature intimately located together. A particular example is chalcopyrite (CuFeS₂) and pyrite (FeS₂), which has a lower value than chalcopyrite, which are often found together in the same mineral grains. Due to the very small grain sizes that occur it is often very difficult to identify and separate such valuable and non-valuable sulphide minerals from each other in mined material.

The applicant has been involved in a research and development program that is based on the use of microwave energy to heat valuable minerals within particles of mined material. Microwave energy includes electric field and magnetic field components that cause direct heating of particles of mined material. The direct heating provides information on the particles that provides a basis for sorting the particles into a stream of relatively valuable particles and a stream of less valuable particles. The sorting step reduces the amount of the particles that are carried forward for further processing to recover valuable material from the particles. The direct heating also provides an opportunity to cause structural alteration, such as microcracking, of the particles that facilitates downstream processing of the particles to recover valuable material from the particles.

The above description is not to be understood as an admission of the common general knowledge in Australia or elsewhere.

### SUMMARY OF THE DISCLOSURE

According to the present invention there is provided a method of processing mined material, such as mined ore, including a step of exposing mined material to an alternating magnetic field and directly or indirectly assessing the electrical conductivity of mined material in the alternating magnetic field in order to determine whether there is valuable material in the mined material.

The invention is based on a realization that valuable material in mined material is often more electrically conductive material than non-valuable material and therefore directly or indirectly assessing the electrical conductivity of mined material can provide an indication of whether there is valuable material in the mined material.

The invention is also based on a realisation that exposing mined material to an alternating magnetic field is a viable option for directly or indirectly assessing electrical conductivity of the mined material.

The term “directly or indirectly assessing the electrical conductivity” is understood herein to include options that directly measure the electrical conductivity and other options that measure or monitor parameters that are related to the electrical conductivity. Specifically, the present invention is not limited to direct measurement of electrical conductivity.

The method may include selecting the alternating magnetic field to induce currents in any valuable material in the mined material to cause indirect heating of valuable material and exposing the mined material to the selected alternating magnetic field and indirectly heating any valuable material and monitoring temperature increases in order to determine whether there is valuable material in the mined material. This is an indirect option for assessing electrical conductivity of the mined material.

The applicant has found that an alternating magnetic field, for example magnetic fields alternating at frequencies in a range of 1 kHz to 100 MHz, can indirectly induce electric currents in mined material which are sufficient to cause a measurable increase in temperature which is related to the type and the amount of valuable material in the mined material.

More particularly, the applicant has found that an alternating magnetic field can indirectly induce electric fields in particles of mined material which in turn induce electric currents in the form of eddy currents in the electrically conductive materials in the particles, with the electric currents causing sufficient selective ohmic heating of the electrically conductive materials in particles in accordance with Joules First Law (i.e. the amount of heat (Q) produced in a specified time is the current squared (I²) multiplied by the electrical resistance (R) of the materials in the particles and the time period) to make it possible to determine whether there is valuable material in the mined material. Thus, the alternating magnetic field allows an increase in selectivity of heating of materials in particles of mined material based solely on properties such as the electrical conductivity and skin depth of valuable materials such as valuable minerals in the particles. The eddy currents that are induced by the alternating magnetic field are directly related to the electrical conductivity of the materials in the particles and therefore only electrical conductive materials, such as chalcopyrite, in the particles are heated and non-electrical conductive or lower electrical conductive materials such as clay and quartz in the particles are not heated at all or at least not to the same extent. There are two mechanisms here. First of all it is necessary that the valuable material be conductive in order to be associated with a substantial current. Second, it is important that the valuable material not be an extremely good conductor as the material will not generate sufficient ohmic heat. Also the smaller the size of the mined material the better will be the volumetric heating. If the mined material is magnetic e.g. ferromagnetic, such as magnetite, the particles are heated strongly with power loss directly proportional to the magnetic permeability. The magnitude of the eddy currents induced is directly proportional to the magnetic field intensity, magnetic field frequency and also electrical conductivity.

The benefits of indirect heating are that the energy is only generated in materials in particles of mined material that are electrically conductive (which changes with frequency) e.g. chalcopyrite has an electrical conductivity of 20-1000 S/m and quartz has a conductivity of 10e⁻¹⁴ S/m demonstrating the orders of magnitude differences between these two materials, one valuable and the other less valuable, often found together in copper-containing mined material which can be exploited to separate particles containing different amounts of these materials. In other words, the present invention makes it possible to discriminate between specific metal sulphide minerals and selectively identify particles containing specific minerals. As noted above, the present invention is not confined to copper-containing mined material and is applicable to a wide range of materials where differences in electrical conductivity of valuable and non-valuable components of the materials make it possible to discriminate between these components.

The method may include selecting the frequency of the magnetic field to optimise heating of valuable materials in mined material. The selection may include operating at multiple magnetic field frequencies. As electrical conductivity and therefore skin depth (or the depth to which currents are induced in particles) varies with magnetic field frequency, an optimum frequency can be determined for each valuable mineral or other valuable constituent material in a mined material in order to make it possible to allow mined material to be heated selectively compared to non-valuable material in mined material. In materials that are insulators, eddy currents are not induced. In materials that are highly conductive, eddy currents are induced but there is insufficient electrical resistance in the materials to produce heat in accordance with Joules First Law. In addition, the inventors have found that (a) electrical conductivity s for each valuable mineral varies greatly between and within mineral classifications depending on the electronic structure and mineral chemistry and (b) the size of the valuable grains within the bulk ore influence the ability to induce currents and therefore generate heating of grains and thus ore particles. These two factors will therefore influence the frequency selection for optimal heating, which may be different for different grains sizes in the same ore particle.

The method may include selecting the alternating magnetic field frequency in a range of 1 kHz to 100 MHz.

The induced eddy currents create heat through ohmic heating in accordance with Joules First Law and the heat can then be used (a) as a basis to sort particles, for example on the basis of grade of a valuable material or (b) if the rate of rise of temperature is fast enough, to fracture particles into smaller particles or to form micro-cracks that make it easier to fracture particles in downstream processing steps.

The method may include selecting the alternating magnetic field to induce currents in any valuable material in the mined material and exposing the mined material to the selected alternating magnetic field and monitoring an operating parameter, such as electrical current that generates the alternating magnetic field and is responsive to electrical conductivity of the mined material, to determine whether there is valuable material in the mined material. This is an indirect option for assessing electrical conductivity of the mined material. Other examples of relevant operating parameters are the resonant frequency and quality factor of the system.

In the method described in the preceding paragraph, the electrical current that is drawn from the generator to create the alternating magnetic field is related to the electrical conductivity of the mined material and therefore an indication of whether there is valuable material in the mined material. The method described in the preceding paragraph does not depend on determining increases in temperatures and comparing temperatures of particles. This is important in situations where the feed mined material is already quite hot after being exposed to high temperatures in stockpiles prior to being processed in the method.

Advantages of the method and apparatus of the invention include:
Reduction of issues around EMC and health and safety
Potentially massively enhanced selectivity over microwave direct heating options leading to lower energy consumption and greater range of ore applicability
Heating of certain valuable mineral phases which are electrically conductive via an induced electric field
No heating of water in particles.
Availability of high power equipment off the shelf at lower costs.
Less sensitivity to conductive minerals creating false positives in sorting particles.
Reduces requirement for feed preparation as low energy is consumed in processing barren materials.
Reduction of arcing possibilities through no electric field breakdown as only magnetic field employed.
The energy draw for generating the alternating magnetic field is proportional to the concentration of the valuable material in the mined material exposed to the alternating magnetic field and hence energy consumption is low in situations where there is barren material and higher when there is valuable material being processed.
Use of HDPE or other plastic materials and not expensive ceramics in the apparatus reduces apparatus costs compared to microwave apparatus direct heating options.

The mined material may be in any suitable form.

Typically, the mined material is in the form of particles.

The term “particle” as used herein may be understood by some persons skilled in the art to be better described as “fragments”. The intention is to use both terms as synonyms.

The term “particle” includes any size particles, including sub-micron particles.

The particles may be transported in a suitable fluid. For example, the particles may be transported in water, for example in the form of a slurry.

The method may include selecting the frequency of the alternating magnetic field to optimise the heating of valuable materials in the mined material when compared to non-valuable materials in the mined material to facilitate discriminating between the valuable and non-valuable materials in the mined material.

For example, in the case of mined material that contains valuable chalcopyrite and non-valuable pyrite and other non-valuable materials such as clay and quartz, the method may include selecting the frequency of the alternating magnetic field to optimise the heating of chalcopyrite compared to the other materials in the mined material.

The method may include selecting the alternating magnetic field to cause structural alteration of the mined material.

The method may include selecting the alternating magnetic field to cause structural alteration of particles of the mined material as a result of differences in thermal expansion of minerals within particles causing regions of high stress/strain within particles and micro-cracking or other physical changes within particles.

There are different options for downstream processing of mined material.

In some instances, the options may include particle by particle assessment of the impact of indirect heating on particles, for example by thermal analysis of individual particles and comparison of the results for particles.

In other instances, the options may include a bulk assessment of the impact of indirect heating of a plurality of particles, for example by bulk thermal analysis to assess a bulk temperature of the plurality of particles.

The method may include thermally analysing indirectly heated particles to detect temperature differences between particles.

The method may include sorting particles on the basis of the results of the temperature differences between particles or the bulk temperature increase of the plurality of particles.

The sorting step may be based on grade of a valuable material in the particles and include separating the particles into a stream of valuable particles and a stream of less valuable particles based on grade. The sorting step may be based on any other suitable property of the particles.

The method may include processing the stream of valuable particles to recover valuable material from the particles. The processing steps may include heap leaching the particles. The processing steps may include forming a concentrate of the particles containing the valuable material and smelting or other recovery steps.

According to the present invention there is also provided an apparatus for processing mined material, such as mined ore, including a magnetic field station that is adapted to expose mined material to an alternating magnetic field that induces currents in mined material that are related to the electrical conductivity of valuable material in the mined material.

The magnetic field station may be adapted to monitor an operating parameter, such as electrical current for generating the alternating magnetic field and/or the resonant frequency or quality factor of the system, to assess the electrical conductivity of valuable material in the mined material.

The apparatus may include a thermal analysis station for detecting temperature increases in mined material from the magnetic field station.

The apparatus may include a sorter for sorting the mined material on the basis of the bulk temperature increase of the mined material or temperature differences between particles of the mined material.

The magnetic field station may include an assembly, such as a conveyor belt or belts, for transporting the mined material through the station.

The magnetic field station may be adapted to expose the mined material to the alternating magnetic field as the mined material moves in a downwardly directed path. For example, the station may be adapted to expose the mined material to the alternating magnetic field as the mined material in a free-fall vertical path.

According to the present invention there is also provided a method for recovering valuable material, such as a valuable metal, from mined material, such as mined ore, including processing mined material according to the method described above and thereafter processing mined material containing valuable material and recovering valuable material.

### BRIEF DESCRIPTION OF THE DRAWINGS

The present invention is described further by way of example with reference to the accompanying drawings, of which:

FIG. 1 is a schematic diagram which illustrates one embodiment of an apparatus for processing mined material in accordance with the present invention; and

FIG. 2 is a diagram that shows the model geometry for induction heating computer modeling work.

### DESCRIPTION OF EMBODIMENT

The embodiment is described in the context of a method of recovering a valuable metal in the form of copper from low grade copper-containing ores in which the copper is present as chalcopyrite and the ores also contain a non-valuable metal sulphide in the form of pyrite and gangue, typically quartz. It is emphasised that the present invention is not confined to copper-containing mined material and is applicable to a wide range of materials where differences in electrical conductivity of valuable and non-valuable components of the materials make it possible to discriminate between these components. An objective, although not the only objective, of the method in this embodiment is to discriminate between chalcopyrite and pyrite minerals. In situations where the chalcopyrite and pyrite minerals are in separate particles, the minerals can be separated into two streams. The separated chalcopyrite particles can then be processed as required to recover copper from the particles. Separating the chalcopyrite particles and the pyrite particles prior to downstream recovery steps significantly increases the average grade of the material being processed in these steps. In situations where the chalcopyrite and pyrite minerals are in the same particles, the ratio of gangue to chalcopyrite to pyrite for each particle can be determined so that an “intelligent” decision regarding the net economic worth of that particle and a preferred downstream recovery option can be made. For example, particles with high copper and high pyrite could be separated into a new stream for blending or extraction using a more conducive approach (e.g. leaching).

The present invention is particularly, although not exclusively, applicable to treating low grade mined material.

The term “low” grade is understood herein to mean that the economic value of the valuable material, such as a metal, in the mined material is only marginally greater than the costs to mine and recover and transport the valuable material to a customer.

In any given situation, the concentrations that are regarded as “low” grade will depend on the economic value of the valuable material and the mining and other costs to recover the valuable material at a particular point in time. The concentration of the valuable material may be relatively high and still be regarded as “low” grade. This is the case with iron ores.

In the case of valuable material in the form of copper sulphide minerals, currently “low” grade ores are run-of-mine ores containing less than 1.0% by weight, typically less than 0.6 wt. %, copper in the ores. Sorting ores having such low concentrations of copper from barren particles is a challenging task from a technical viewpoint, particularly in situations where there is a need to sort very large amounts of ore, typically at least 10,000 tonnes per hour, and where the barren particles represent a smaller proportion of the ore than the ore that contains economically recoverable copper.

The term “barren” particles, when used in the context of copper-containing ores, are understood herein to mean particles containing minerals with no copper (such as pyrite) or very small amounts of copper that cannot be recovered economically from the particles.

The term “barren” particles when used in a more general sense in the context of valuable materials is understood herein to mean particles with no valuable material or amounts of valuable material that cannot be recovered economically from the particles.

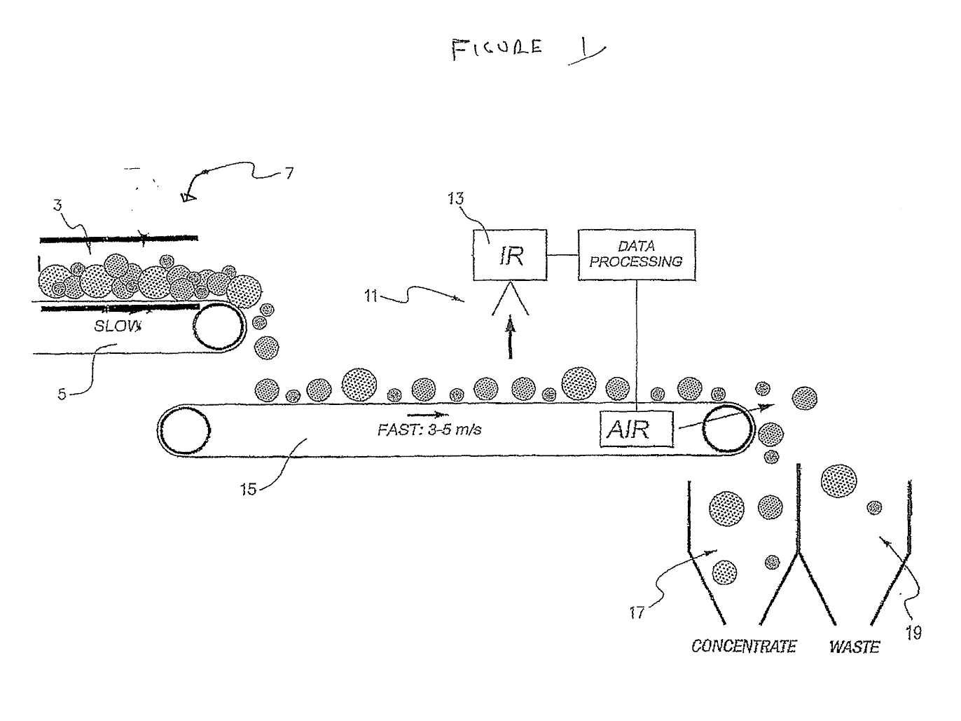

With reference to the embodiment of the processing apparatus in accordance with the invention shown in FIG. 1, a feed material in the form of ore particles 3 that have been crushed by a primary crusher (not shown) to a particle size of 10-25 cm are transported via a conveyor 5 (or other suitable transfer means) through an induction heating coil 7 and are heated in the coil. The induction heating coil 7 may be any suitable induction heating coil 7 that is capable of generating an alternating and homogeneous magnetic field having a required frequency, typically in a range of 1 kHz to 100 MHz, to heat the ore particles 3 as a consequence of the electrical conductivity of the chalcopyrite in the ore particles 3 to a required extent for downstream thermal analysis and at a required throughput.

As indicated above, the term “particle” as used herein may be understood by some persons skilled in the art to be better described as “fragments”. The intention is to use both terms as synonyms.

The induction heating coil 7 induces localised heating of the materials in the particles depending on the electrical conductivity of the materials in the particles. In particular, subject to appropriate selection of the operating magnetic field frequency of the induction heating coil 7, the particles are heated to different extents depending on the materials in the particles. The inventors have found that it is possible to operate the induction heating coil 7 so that particles having relatively small concentrations of chalcopyrite are heated to a greater extent than particles having no chalcopyrite and high concentrations of pyrite and other non-valuable materials. As noted above, the inventors have found that (a) electrical conductivity for each valuable mineral varies greatly between and within mineral classifications depending on the electronic structure and mineral chemistry and (b) the size of the valuable grains within the bulk ore influence the ability to induce currents and therefore generate heating of grains and thus ore particles. These two factors are examples of factors that influence the frequency selection for optimal heating, which may be different for different grains sizes in the same ore particle.

The basis of thermal analysis in this embodiment is that particles that contain chalcopyrite will become hotter than particles containing no chalcopyrite but high concentrations of pyrite and other non-valuable materials, i.e. barren particles, only when exposed to induction heating in the induction heating coil 7.

The particles that pass through the induction heating coil 7 drop from the end of the conveyor belt 5 onto a lower conveyor belt 15 and are transported on this belt through an infra-red radiation detection station 11 at which the particles are viewed by an infra-red camera 13 (or other suitable thermal detection apparatus) and are analysed thermally. The conveyor belt 15 is operated at a faster speed than the conveyor belt 5 to allow the particles to spread out along the belt 15. This is helpful in terms of the downstream processing of the particles.

The spacing between the stations 7 and 11 is selected having regard to the belt speed to allow sufficient time, typically at least 5 seconds, for the particles to be heated uniformly within each particle.

Advantageously, the upstream processing conditions are selected so that the s particles have sufficient retained heat for thermal analysis without additional heating of the particles being required. If additional heating is required, it can be provided by any suitable means.

In one mode of operation the thermal analysis is based on distinguishing between particles that are above and below a threshold temperature. The particles can then be categorised as “hotter” and “colder” particles. The temperature of a particle is related to the amount of copper minerals in the particle. Hence, particles that have a given particle size range and are heated under given conditions will have a temperature increase to a temperature above a threshold temperature “x” degrees if the particles contain at least “y” wt. % copper. The threshold temperature can be selected initially based on economic factors and adjusted as those factors change. Barren particles will generally not be heated on exposure to microwave energy to temperatures above the threshold temperature.

Once identified by thermal analysis, the hotter particles are separated from the colder particles and the hotter particles are thereafter processed to recover copper from the particles. Depending on the circumstances, the colder particles may be processed in a different process route to the hotter particles to recover copper from the colder particles.

The particles are separated by being projected from the end of the conveyor belt 15 and being deflected selectively by compressed air jets (or other suitable fluid jets, such as water jets) as the particles move in a free-fall trajectory from the belt 15 and thereby being sorted into two streams 17, 19. In this connection, the thermal analysis identifies the position of each of the particles on the conveyor belt 15 and the air jets are activated a pre-set time after a particle is analysed as a particle to be deflected.

Depending on the particular situation, the gangue particles may be deflected by air jets or the particles that contain copper above a threshold concentration may be deflected by air jets.

The hotter particles become a concentrate feed stream 17 and are transferred for downstream processing, typically including milling, flotation to form a concentrate, and then further processing to recover copper from the particles.

The colder particles may become a by-product waste stream 19 and are disposed of in a suitable manner. This may not always be the case. The colder particles have lower concentrations of copper minerals and may be sufficiently valuable for recovery. In that event the colder particles may be transferred to a suitable recovery process, such as leaching.

It is noted that the embodiment described in relation to FIG. 1 is not the only possible embodiment of the invention. For example, the invention is not confined to transporting particles through an induction heating coil 7 on a conveyor belt 15. Any suitable transport option may be used. Specifically, the invention extends to allowing particles to move in a downward path through the induction heating coil 7. In addition, by way of further example, the invention is not confined to particle by particle assessment of the impact of indirect heating of particles and extends to making a bulk temperature assessment of a plurality of particles.

The present invention is based on computer modeling and heating trials carried out by the inventors. This work is described below.

### Induction Heating Computer Modeling Work

The objective of the study was to identify and determine the potential for inductive heating of selected materials of interest (chalcopyrite and pyrite and quartz (representative of gangue)) in ore particles. The simulations were performed using COMSOL Multiphysics 4.3.a, AC/DC Module.

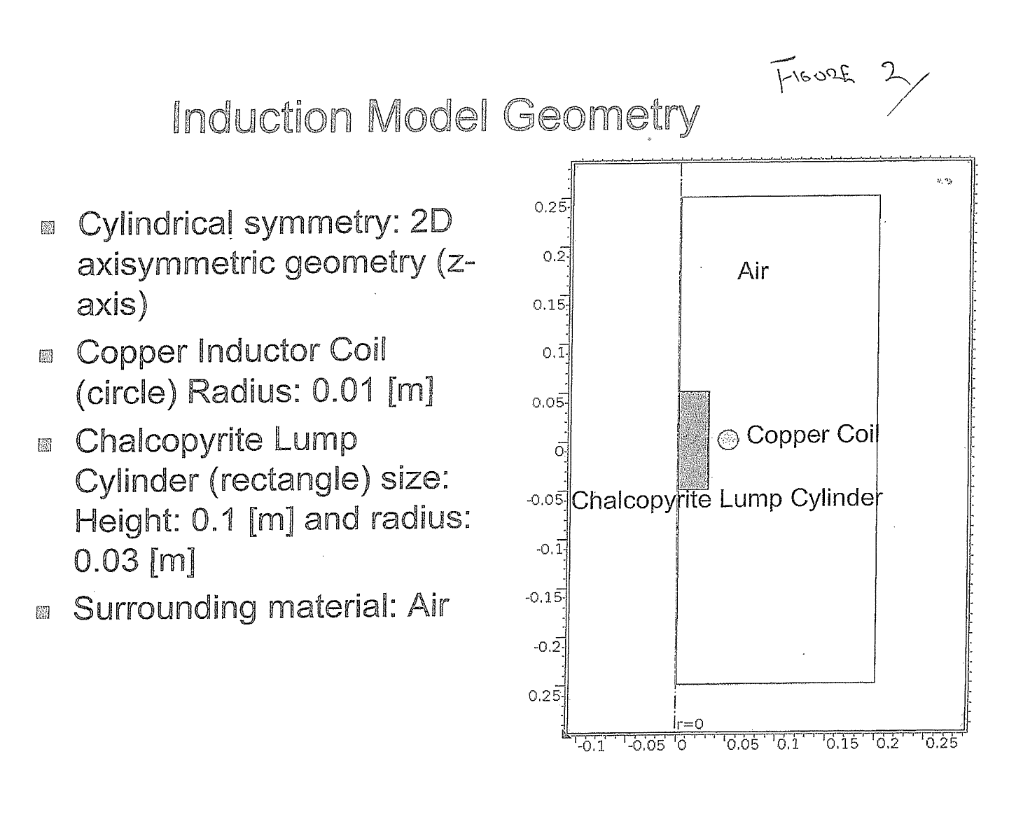

The model consists of an alternating current (AC) coil surrounding an ore cylinder (core) made of quartz with mineral insertions (chalcopyrite and pyrite and quartz), all surrounded by air. The coil induces eddy currents in the ore cylinder. A 2D axisymmetric geometry (about the z-axis) was used due to the cylindrical symmetry. The model geometry is shown in FIG. 2.

The physics used in the model is Magnetic Fields, with a Frequency Domain study type.

Boundary conditions used included zero magnetic flux through the exterior air boundary (vector potential=zero) and a symmetry boundary condition on the z-axis.

A Single-Turn Coil Domain model was used to excite the coil with the coil current of I_{coil}=1.5 kA constant.

The material properties used in the model were:

| | Electrical | | |
|---|---|---|---|
| | conductivity | Relative | Relative |
| Material | [S/m] | Permittivity | permeability |
| Air (Surrounding domain) | 0 | 1 | 1 |
| Copper (AC coil) | 6*10⁷ | 1 | 1 |
| Chalcopyrite (Mineral | 196.0784314 | 1 | 1.000200 |
| insertion) | | | |
| Pyrite (Mineral insertion) | 20 | 1 | 1.001000 |
| Quart (Gangue) | 10⁻¹⁴ | 4.2 | 0.999985 |

The material properties are based on the following literature values form the sources mentioned bellow:

| | | | | Chosen | Mass | | | |
|---|---|---|---|---|---|---|---|---|
| | | Density | Susceptibility | Susceptibility | Susceptibility χₘ | Relative | | Conductivity |
| | Chemical | (10³ kg | χᵥ (volumetric | χᵥ (volumetric | (χᵥ/p) (10⁻⁸ m⁻³ | Permeability | Resistivity | [S/m] = |
| Mineral | Formula | (m⁻³) | (10⁻⁶ SI) | SI) | kg⁻¹) | μ/μ0 = 1 + χᵥ | (W * m) | 1/Resistivity |
| Chalcopyrite | CuFeS2 | 4.2 | 23-400 | 0.000200 | 0.55-10 | 1.000200 | 0.0051 | 196.0784 |
| Pyrite | FeS2 | 5.02 | 35-5000 | 0.001000 | 1-100 | 1.001000 | 0.05 | 20 |
| Quartz | SiO2 | 2.65 | −13-17 | −0.000015 | −0.5-0.6 | 0.999985 | | 10⁻¹⁴ |

1. Susceptibility data from: Rock physics & phase relations: a handbook of physical constants By Thomas J. Ahrens
2. The electrical resistivity of galena, pyrite, and chalcopyrite, by D. F. Pridmore and R. T. Shuey Department of Geology and Geophysics, University of Utah; Salt Lake City, Utah 84112

The simulations were performed with chalcopyrite on its own, pyrite on its own, quartz on its own, and chalcopyrite, pyrite and quartz together at 11 magnetic field frequencies from 50 Hz to 2 MHz.

The simulations established that, based on the data for the materials used in the model, with appropriate selection of magnetic field frequencies, typically high frequencies, it is possible to selectively heat chalcopyrite in comparison to pyrite and quartz. This is an indication that induction heating can discriminate between a valuable material and non-valuable materials.

### Induction Heating Trials

The objective of the heating trials was to determine the potential for inductive heating of selected materials (chalcopyrite and pyrite and clay) of interest, real ore fragments, and previously assayed pulverised ore powders. The test work was performed using a Cheltenham Low Frequency Induction Heater, type TR1 operating at 166 kHz.

The real ore fragments and material samples weighed approximately 8.0 g. For the pulverised ore powders, approximately 5.0 g of each powder was weighed out. The test materials were imaged before heating using an NEC AVIO H2640 infra-red thermal imaging camera. The materials were then transferred to a glass test tube. Powders were tapped down to reduce the volume of air and improve thermal conduction between powder grains.

The induction heating system was switched on to establish auto-matching of frequency to the coil, 166 kHz (this frequency is a function of the coil and not of the load which is placed within it). The glass tube was then clamped in the coil. The test materials were treated for 20 s (ore and mineral fragments) or 40 s (powders) at 0.7 kW (this is an under loaded condition for the induction system, outside the ideal working range).

The timer function on the system was used to maintain constant treatment time across all test materials. The tube was then removed from the coil and the test material transferred to the imaging area where change in temperature was recorded using the thermal imaging camera. Post treatment imaging was taken at 1 frame/s for 30 s. The time from end of treatment to imaging was approximately 10 s.

Table 1 below shows temperature data for certain test materials.

TABLE 1

| Mineral | ΔTₐᵥ ° C. | ΔTₘₐₓ ° C. |
|---|---|---|
| Pyrite | 5 | 10 |
| Chalcopyrite | 32 | 65 |
| Clay | 3 | 8 |

It is evident from Table 1 that there were significant differences in temperature between valuable chalcopyrite and non-valuable pyrite and clay.

Moreover, the conclusions from the induction heating trial are as follows:
Tests clearly indicate a degree of inductive heating
Chalcopyrite heated more rapidly than pyrite
   opportunity for improved selectivity
Clay type mineral (phlogopite) had little thermal response at this frequency
   possibility to remove inherent noise in thermal responses of MW systems due to moisture content of ores
Induction heating of real ore fragments indicate selectivity between barren, middling and valuable grades
Modelling indicates that thermal responses of valuable phases will be much greater at higher frequencies
Large reduction in energy input required compared to trials

Whilst a specific apparatus and method embodiment have been described, it should be appreciated that the apparatus and method may be embodied in many other forms.

The embodiment is described in the context of a method and an apparatus for recovering a valuable metal in the form of copper from a low grade copper-containing OTC in which the copper is present in copper-containing minerals such as chalcopyrite Io and the ore also contains non-valuable gangue.

It is noted that the present invention is not confined to copper-containing ores and to copper as the valuable material to be recovered.

In the claims which follow, and in the preceding description, except where the context requires otherwise due to express language or necessary implication, the word “comprise” and variations such as “comprises” or “comprising” are used in an inclusive sense, i.e. to specify the presence of the stated features but not to preclude the presence or addition of further features in various embodiments of the apparatus and method as disclosed herein.

1. A method of processing mined material including a step of exposing mined material to an alternating magnetic field and directly or indirectly assessing the electrical conductivity of mined material in the alternating magnetic field in order to determine whether there is valuable material in the mined material.

2. The method defined in claim 1 includes selecting the alternating magnetic field to induce currents in any valuable material in the mined material and cause indirect heating of valuable material and exposing the mined material to the selected alternating magnetic field and indirectly heating any valuable material and monitoring temperature increases in order to determine whether there is valuable material in the mined material.

3. The method defined in claim 2 includes selecting the frequency of the alternating magnetic field to optimise heating of valuable materials in the mined material when compared to non-valuable materials in the mined material to facilitate discriminating between the materials in the mined material.

4. The method defined in claim 3 wherein in the case of mined material that contains valuable chalcopyrite and non-valuable pyrite and other non-valuable materials, the method includes selecting the frequency of the alternating magnetic field to optimise heating of chalcopyrite compared to the other materials in the mined material.

5. The method defined in claim 1 includes selecting the alternating magnetic field to induce currents in any valuable material in the mined material and exposing the mined material to the selected alternating magnetic field and monitoring an operating parameter, such as electrical current that generates the alternating magnetic field and is responsive to electrical conductivity of the mined material and/or the resonant frequency or quality factor of the system, to determine whether there is valuable material in the mined material.

6. The method defined in claim 1 includes selecting the alternating magnetic field to cause structural alteration of the mined material.

7. The method defined in claim 6 includes selecting the alternating magnetic field to cause structural alteration of the mined material as a result of differences in thermal expansion of minerals within particles of the mined material causing regions of high stress/strain within particles and micro-cracking or other physical changes within particles.

8. An apparatus for processing mined material including a magnetic field station that is adapted to expose mined material to an alternating magnetic field that induces currents in mined material that are related to the electrical conductivity of valuable material in the mined material.

9. The apparatus defined in claim 8 wherein the magnetic field station is adapted to monitor an operating parameter, such as electrical current for generating the alternating magnetic field and/or the resonant frequency or quality factor of the system, to assess the electrical conductivity of valuable material in the mined material.

10. The apparatus defined in claim 8 includes a thermal analysis station for detecting temperature increases in mined material from the magnetic field station.

11. The apparatus defined in claim 8 includes a sorter for sorting the mined material on the basis of the bulk temperature increase of the mined material or temperature differences between particles of the mined material.

12. A method for recovering valuable material from mined material, including processing mined material according to the method defined in claim 1 and thereafter processing mined material containing valuable material and recovering valuable material.

13. The method defined in claim 1 wherein the mined material is mined ore.

14. The method defined in claim 12 wherein the mined material is mined ore.
